(19) United States
(12) Patent Application Publication
(10) Pub. No.: US 2026/0114764 A1
(43) Pub. Date: Apr. 30, 2026

(54) DETECTION DEVICE

(71) Applicant: Japan Display Inc., Tokyo (JP)

(72) Inventor: Masahiro TADA, Tokyo (JP)

(21) Appl. No.: 19/363,237

(22) Filed: Oct. 20, 2025

(30) Foreign Application Priority Data
Oct. 29, 2024 (JP) 2024-190102

(51) Int. Cl. A61B 5/1455; A61B 5/00; A61B 5/01

(52) U.S. Cl. CPC A61B 5/14551; A61B 5/01; A61B 5/6826; A61B 2560/0219; A61B 2562/0219

(57) ABSTRACT

According to an aspect, a detection device includes: an optical sensor and a light source; and a light-transmitting dome-shaped or spherical frustum-shaped housing that covers the optical sensor and the light source. The optical sensor and the light source are arranged on the same plane in plan view.

### CROSS-REFERENCE TO RELATED APPLICATION

This application claims the benefit of priority from Japanese Patent Application No. 2024-190102 filed on Oct. 29, 2024, the entire contents of which are incorporated herein by reference.

### BACKGROUND

### 1. Technical Field

What is disclosed herein relates to a detection device.

### 2. Description of the Related Art

Optical sensors capable of detecting fingerprint patterns and vein patterns are known (for example, Japanese Patent Application Laid-open Publication No. 2009-032005 (JP-A-2009-032005)). Such optical sensors are each built into a wearable device, such as a smartwatch, a wristwatch, or a wristband, and used for acquiring biometric information, such as pulse waves.

However, in detection devices using an optical sensor of JP-A-2009-032005, measurement accuracy may be degraded by effects of body motion or external light.

For the foregoing reasons, there is a need for a detection device capable of easily acquiring accurate biometric information.

### SUMMARY

According to an aspect, a detection device includes: an optical sensor and a light source; and a light-transmitting dome-shaped or spherical frustum-shaped housing that covers the optical sensor and the light source. The optical sensor and the light source are arranged on the same plane in plan view.

### BRIEF DESCRIPTION OF THE DRAWINGS

FIG. 1 is a schematic sectional view illustrating a detection device according to a first embodiment in a state of being accommodated in the navel of a human body;

FIG. 2 is a schematic sectional view of the detection device according to the first embodiment;

FIG. 3 is a block diagram illustrating a configuration example of a control circuit of the detection device;

FIG. 4 is a plan view of a section along IV-IV′ in FIG. 2;

FIG. 5 is a plan view of a section along V-V′ in FIG. 2;

FIG. 6 is a plan view of a section along VI-VI′ in FIG. 2;

FIG. 7 is another example of the section along VI-VI′ in FIG. 2 and is a plan view of a detection device according to a second embodiment;

FIG. 8 is a schematic sectional view of a detection device according to a first modification of the second embodiment;

FIG. 9 is a plan view of a section along IX-IX′ in FIG. 8;

FIG. 10 is a schematic sectional view of a detection device according to a second modification of the second embodiment;

FIG. 11 is a plan view of a section along XI-XI′ in FIG. 10;

FIG. 12 is a schematic sectional view illustrating a detection device according to a third embodiment in the state of being accommodated in the navel of the human body;

FIG. 13 is a schematic diagram illustrating a configuration example of a communication antenna of a detection device according to a fourth embodiment;

FIG. 14 is a schematic diagram illustrating a configuration example of a communication antenna of a detection device according to a comparative example;

FIG. 15 is a schematic diagram illustrating a configuration example of a communication antenna of a detection device according to a modification of the fourth embodiment;

FIG. 16 is a sectional view of a detection device according to a fifth embodiment; and

FIG. 17 is a plan view of the detection device according to the fifth embodiment.

### DETAILED DESCRIPTION

The following describes modes (embodiments) for carrying out the present disclosure in detail with reference to the drawings. The present disclosure is not limited to the description of the embodiments given below. Components described below include those easily conceivable by those skilled in the art or those substantially identical thereto. In addition, the components described below can be combined as appropriate. What is disclosed herein is merely an example, and the present disclosure naturally encompasses appropriate modifications easily conceivable by those skilled in the art while maintaining the gist of the present disclosure. To further clarify the description, the drawings may schematically illustrate, for example, widths, thicknesses, and shapes of various parts as compared with actual aspects thereof. However, they are merely examples, and interpretation of the present disclosure is not limited thereto. The same component as that described with reference to an already mentioned drawing is denoted by the same reference numeral through the present disclosure and the drawings, and detailed description thereof may not be repeated where appropriate.

In the present disclosure, in expressing an aspect of disposing another structure on or above a certain structure, a case of simply expressing “on” includes both a case of disposing the other structure immediately on the certain structure so as to contact the certain structure and a case of disposing the other structure above the certain structure with still another structure interposed therebetween, unless otherwise specified.

### First Embodiment

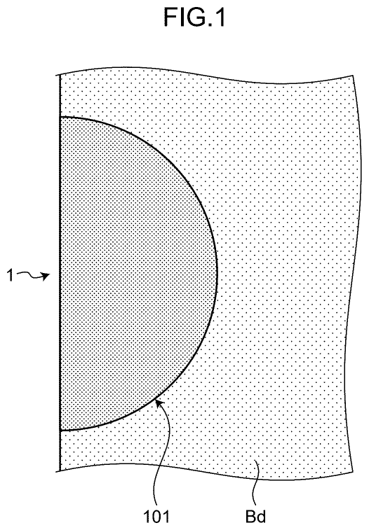

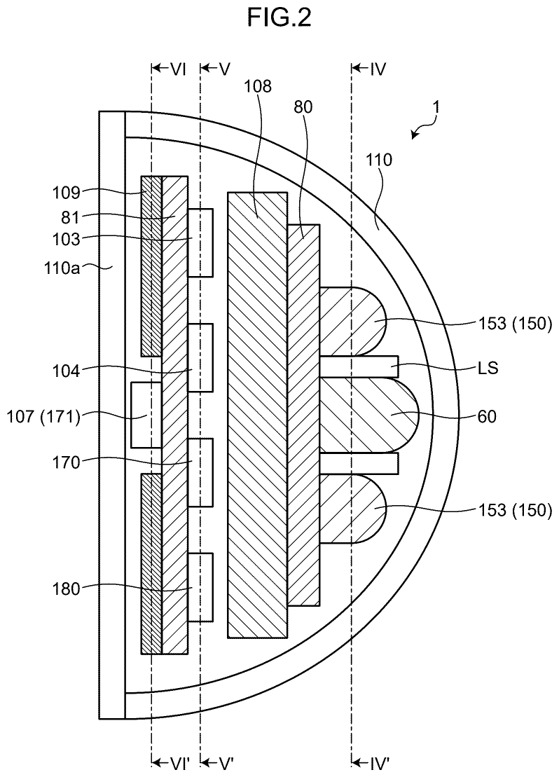

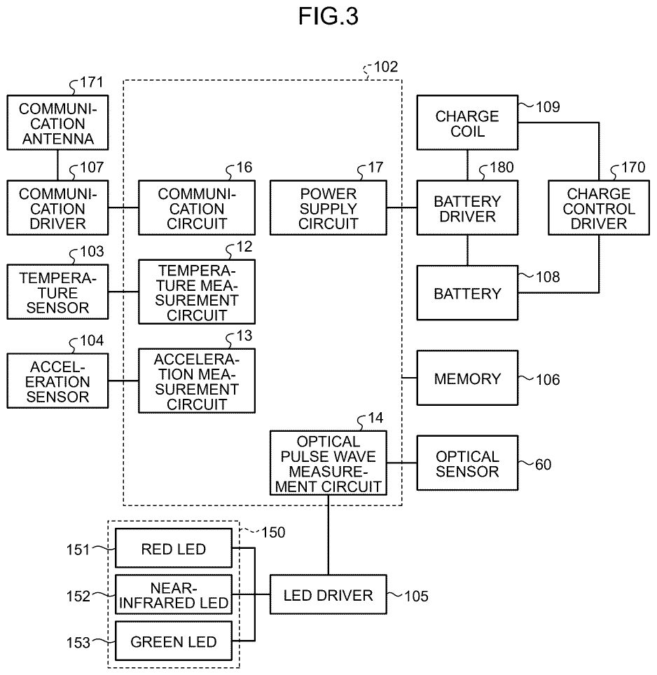

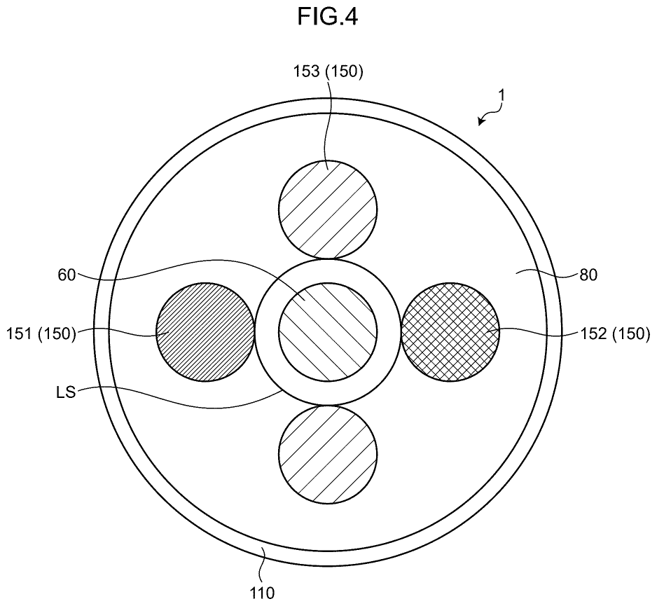

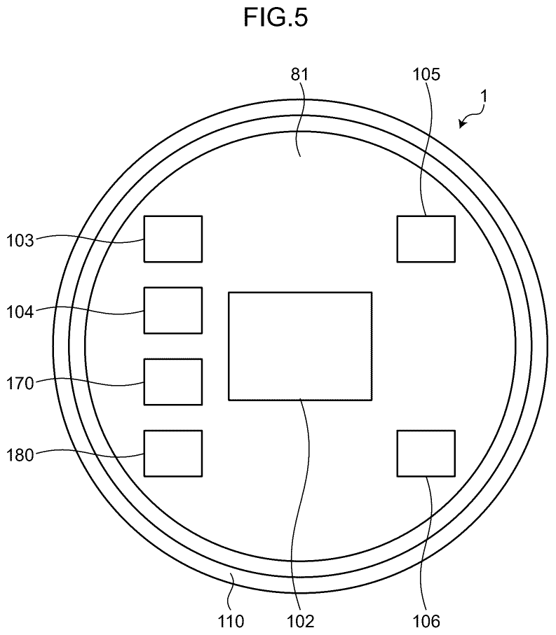

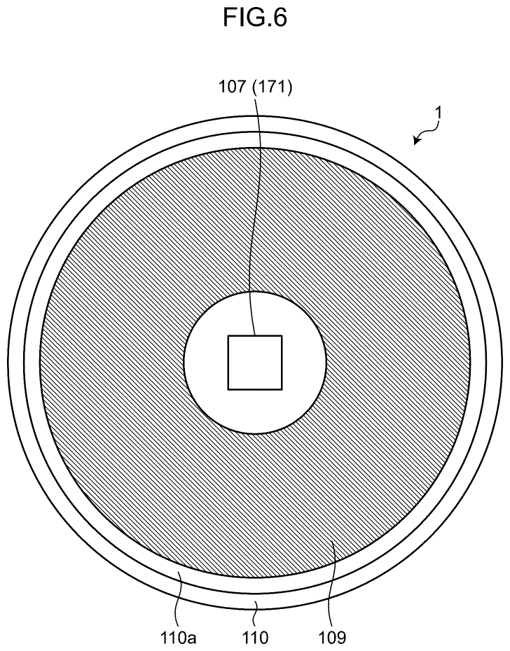

FIG. 1 is a schematic sectional view illustrating a detection device according to a first embodiment in a state of being accommodated in the navel of a human body. FIG. 2 is a schematic sectional view of the detection device according to the first embodiment. FIG. 3 is a block diagram illustrating a configuration example of a control circuit of the detection device. FIG. 4 is a plan view of a section along IV-IV′ in FIG. 2. FIG. 5 is a plan view of a section along V-V′ in FIG. 2. FIG. 6 is a plan view of a section along VI-VI′ in FIG. 2.

As illustrated in FIG. 1, a detection device 1 according to the first embodiment is a device that can be worn on and removed from an umbilical fossa 101 of a human body Bd. The detection device 1 is attached to the umbilical fossa 101. The detection device 1 can detect biometric information on a living body from the umbilical fossa 101 to which the detection device 1 is attached. The umbilical fossa 101 is an example of a measurement target. The measurement target is the living body or a part of the living body and is an object to be measured.

As illustrated in FIG. 2, the detection device 1 includes a housing 110, a light source 150, an optical sensor 60, and circuit boards 80 and 81.

The housing 110 has a dome shape and a light-transmitting property. The housing 110 covers the optical sensor 60 and the light source 150. The housing 110 is made of a transparent resin, for example.

This configuration allows the detection device 1 to securely accommodate the housing 110 in the umbilical fossa 101, thus being capable of improving adhesion.

The housing 110 has a bottom surface 110a. The bottom surface 110a has a light-blocking property.

The circuit boards 80 and 81 each employ a flexible printed circuit board (PCB). The circuit boards 80 and 81 may each be formed using a substrate generally employed as a flexible printed circuit (FPC).

The detection device 1 further includes a charge control driver 170, a battery driver 180, a battery 108, a charge coil 109, a control circuit 102, a temperature sensor 103, an acceleration sensor 104, a light-emitting diode (LED) driver 105, a memory 106, a communication driver 107, and a communication antenna 171 that are provided in the housing 110.

A detailed configuration example of the charge control driver 170, the battery driver 180, the battery 108, the charge coil 109, the control circuit 102, the temperature sensor 103, the acceleration sensor 104, the LED driver 105, the memory 106, the communication driver 107, and the communication antenna 171 will be described later with reference to FIG. 3.

A plurality of types of light-emitting diodes (LEDs) are used as the light source 150. As illustrated in FIG. 4, the light source 150 includes a red LED 151, a near-infrared LED 152, and a green LED 153. The red LED 151 outputs red light. The near-infrared LED 152 outputs near-infrared light. The green LED 153 outputs green light.

The light source 150 emits the light in the umbilical fossa. The optical sensor 60 is an organic photodiode (OPD), for example. The optical sensor 60 detects light that has been output from the red LED 151, the near-infrared LED 152, and the green LED 153 and reflected in the human body Bd through the umbilical fossa 101. The optical sensor 60 detects changes in intensity of incoming light. The optical sensor 60 is an organic photodiode (OPD), for example, and outputs an electrical signal corresponding to the light emitted thereto. The optical sensor 60 may be a PIN (Positive Intrinsic Negative) photodiode formed of a semiconductor such as silicon.

When the red LED 151 is lit and the near-infrared LED 152 and the green LED 153 are not lit, the red light transmitted through the umbilical fossa 101 is detected by the optical sensor 60. When the near-infrared LED 152 is lit and the red LED 151 and the green LED 153 are not lit, the near-infrared light transmitted through the umbilical fossa 101 is detected by the optical sensor 60. When the green LED 153 is lit and the red LED 151 and the near-infrared LED 152 are not lit, the green light transmitted through the umbilical fossa 101 is detected by the optical sensor 60.

The temperature sensor 103 detects the temperature of the umbilical fossa 101 when the detection device 1 is inserted in the umbilical fossa 101. The temperature sensor 103 detects the temperature of the environment surrounding the housing 110 when the detection device 1 is not inserted in the umbilical fossa 101. The temperature sensor 103 supplies information indicating the detected temperature to the control circuit 102.

The acceleration sensor 104 detects an acceleration applied to the detection device 1. The acceleration sensor 104 supplies information indicating the detected acceleration to the control circuit 102. The detected acceleration values can be used to remove effects of body motion of a user on the detection device 1.

The LED driver 105 drives the red LED 151, the near-infrared LED 152, and the green LED 153 to emit light therefrom.

The memory 106 is a storage that stores therein various types of data. The memory 106 may have a configuration with, for example, a random-access memory (RAM), a read-only memory (ROM), an electrically erasable programmable read-only memory (EEPROM), and the like. The memory 106 stores therein various types of information used in the control circuit 102. The memory 106 may be included in the control circuit 102.

The communication driver 107 is a near-field communication driver, for example, and includes the communication antenna 171. The communication antenna 171 is a Bluetooth low-energy (BLE) communication antenna, for example. The communication driver 107 exchanges signals between the detection device 1 and other devices. The communication driver 107 can transmit data and the like measured by various parts of the detection device 1 to other devices. The communication driver 107 can also receive data and the like transmitted by other devices.

The battery 108 supplies power to various parts of the detection device 1. The battery 108 is a lithium-ion battery, for example.

The charge control driver 170 is electrically coupled to the battery 108 and the charge coil 109. The charge control driver 170 controls charging of the battery 108 by adjusting the power supplied from the charge coil 109.

The battery driver 180 controls the battery 108. The battery 108 is charged by the battery driver 180.

The charge coil 109 is a coil for charging the battery 108. The charge coil 109 is electrically coupled to a rectifier circuit (not illustrated) and magnetically coupled to a power transmission coil of a charger or the like when coming closer thereto, thus receiving an electromagnetic field from the power transmission coil and converting it into an electric current. The charge coil 109 may be shared with a near-field communication (NFC) antenna and capture electromagnetic waves from the surrounding space to absorb energy.

As illustrated in FIG. 2, the battery 108 is provided on a side of the circuit board 80 closer to the bottom surface 110a of the housing 110, and the optical sensor 60 is located on a side of the circuit board 80 closer to the apex of the housing 110.

As illustrated in FIG. 4, the optical sensor 60 and the light source 150 are arranged on the same plane in plan view. The optical sensor 60 is located at the center of the housing 110 in plan view. A plurality of the light sources 150 are arranged around the optical sensor 60 in plan view. The optical sensor 60 is surrounded by a light-blocking wall LS in plan view. The light sources 150, that is, the red LED 151, the near-infrared LED 152, and two green LEDs 153 are each located around the light-blocking wall LS.

With this configuration, by lighting the green LED 153 and measuring an optical change of light incident on the optical sensor 60, a pulse wave can be measured, and a pulse wave interval (R-R interval (RRI)) can be measured from the waveform of the pulse wave.

The red LED 151 and the near-infrared LED 152 are alternately lit, and the difference in light absorption therebetween is used to calculate a blood oxygen level. Since the optical sensor 60 is located in the umbilical fossa 101, artifacts due to external light do not occur as is the case of watch-shaped or finger ring-shaped sensors, and accurate biometric information can be easily acquired.

As illustrated in FIG. 4, the maximum diameter of the housing 110 is 3 cm or smaller in plan view. The maximum diameter of the housing 110 is a size enough to be accommodated in the umbilical fossa 101.

As illustrated in FIG. 5, the acceleration sensor 104 is located on the same plane as the control circuit 102.

By locating the acceleration sensor 104 close to the center of gravity of the human body and measuring the acceleration in three axes, the detection device 1 according to the first embodiment can accurately measure amounts of activity without being affected by arm swing.

By locating the acceleration sensor 104 close to the center of gravity of the human body and measuring the acceleration in three axes, the detection device 1 according to the first embodiment can measure the rate of respiration during sleep from motion of the diaphragm.

As illustrated in FIG. 5, the housing 110 is provided with the control circuit 102 on the back side of the optical sensor 60 and the light source 150 with the circuit board 80 and the battery 108 interposed therebetween.

As illustrated in FIG. 5, the control circuit 102 is located at the center of the circuit board 81, and the LED driver 105, the memory 106, the battery driver 180, the charge control driver 170, the acceleration sensor 104, and the temperature sensor 103 are arranged clockwise in this order so as to surround the control circuit 102.

As illustrated in FIGS. 5 and 6, the charge coil 109 and the communication antenna 171 are located on the back side of the control circuit 102.

As illustrated in FIG. 6, the communication antenna 171 is located at the center of the housing 110 in plan view, and the charge coil 109 is shaped like a doughnut and is located so as to be wound around the communication antenna 171 in plan view.

This configuration enables improvement in charge efficiency and communication speed.

As illustrated in FIG. 3, the control circuit 102 controls the various parts of the detection device 1. The control circuit 102 includes, for example, a microcontroller unit (MCU) and the like. The control circuit 102 is, for example, an integrated circuit (IC), such as a microcontroller, or a programmable logic device (PLD), such as a field-programmable gate array (FPGA). The control circuit 102 includes a temperature measurement circuit 12, an acceleration measurement circuit 13, an optical pulse wave measurement circuit 14, a communication circuit 16, and a power supply circuit 17.

The temperature measurement circuit 12 is electrically coupled to the temperature sensor 103. The temperature measurement circuit 12 acquires body temperature data from the temperature sensor 103.

This configuration allows easy measurement of deep body temperature at the navel. Using the temperature measurement circuit 12 in the umbilical fossa of a newborn as a replacement for an umbilical plug, vital measurements as well as measures against umbilical hernia can be made, thus allowing early detection of respiratory disorders, cardiovascular problems, and the like.

The acceleration measurement circuit 13 is electrically coupled to the acceleration sensor 104. The acceleration measurement circuit 13 obtains the amounts of activity based on acceleration data of the acceleration sensor 104.

The optical pulse wave measurement circuit 14 is coupled to the LED driver 105 and the optical sensor 60. The optical pulse wave measurement circuit 14 measures at least one of a pulse frequency or the blood oxygen level based on detection data of the optical sensor 60.

The communication circuit 16 is coupled to the communication driver 107. The communication circuit 16 transmits measurement results and so forth to an external device.

The external device is, for example, a terminal device such as a smartphone or a tablet computer held by a user of the detection device 1. The terminal device such as the smartphone or the tablet computer includes a display screen.

The detection device 1 can easily cause the external device to output the data received from the detection device 1 through the BLE communication to check the output data.

The power supply circuit 17 is coupled to the battery driver 180. The battery driver 180 is electrically coupled to the battery 108 and the charge coil 109. The power supply circuit 17 controls the charging of the battery 108 and supplies the power from the battery 108 to the various parts.

### Second Embodiment

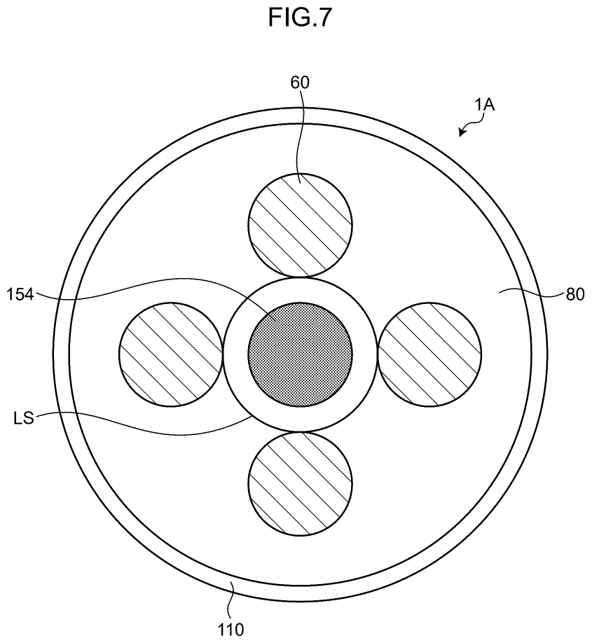

FIG. 7 is another example of the section along VI-VI′ in FIG. 2, and is a plan view of a detection device according to a second embodiment. In the following description, the same components as those described in the embodiment described above are denoted by the same reference numerals, and the description thereof will not be repeated.

A detection device 1A according to the second embodiment includes a light source 154 in which the red LED 151, the near-infrared LED 152, and the green LED 153 are integrated.

The optical sensor 60 and the light source 154 are arranged on the same plane in plan view. The light source 154 is located at the center of the housing 110 in plan view. Four optical sensors 60 are arranged around the light source 154 in plan view. The light source 154 is surrounded by the light-blocking wall LS in plan view. Since the light-blocking wall LS is located between the light source 154 and the optical sensor 60, light directly reaching from the light source 154 to the optical sensor 60 is reduced. The light from the light source 154 reaches the optical sensor 60 through the umbilical fossa 101 of the human body Bd.

This configuration increases the sensor sensitivity of the optical sensor. The detection device 1A according to the second embodiment may reduce the power for driving the light source to the extent that the sensor sensitivity increases. As a result, the detection device 1A according to the second embodiment can extend the interval between charges.

### First Modification of Second Embodiment

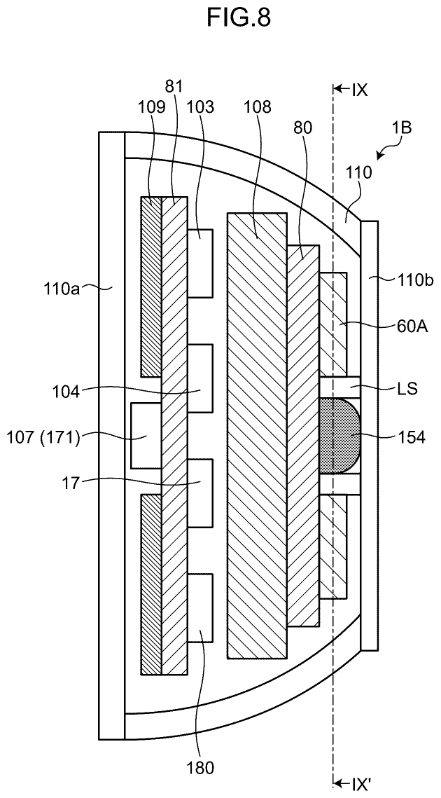

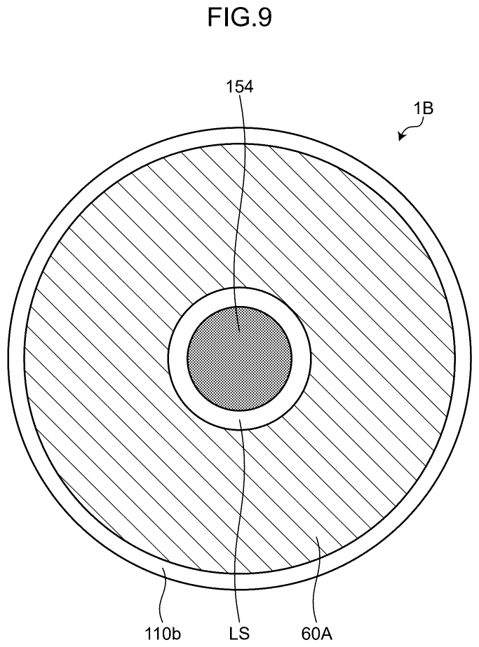

FIG. 8 is a schematic sectional view of a detection device according to a first modification of the second embodiment. FIG. 9 is a plan view of a section along IX-IX′ in FIG. 8. In the following description, the same components as those described in either of the embodiments described above are denoted by the same reference numerals, and the description thereof will not be repeated.

As illustrated in FIG. 9, in a detection device 1B according to the first modification of the second embodiment, the housing 110 has a top surface 110b. The top surface 110b is parallel to the light source 154 and an optical sensor 60A, and the top surface 110b has a flat plate shape. The housing 110 of the detection device 1B according to the first modification of the second embodiment has a spherical frustum shape that is a shape obtained by cutting a sphere by the top surface 110b and the bottom surface 110a parallel to each other. Since the optical sensor 60A is parallel to the top surface 110b, the transmittance of light reaching the optical sensor 60A from the umbilical fossa 101 of the human body Bd through the top surface 110b can be increased. As a result, the sensor sensitivity of the optical sensor 60A increases.

As illustrated in FIG. 9, the optical sensor 60A is a planar sensor shaped like a doughnut. The light source 154 is located at the center of the housing 110 in plan view. The light source 154 is surrounded by the light-blocking wall LS in plan view. The light-blocking wall LS is located between the light source 154 and the optical sensor 60A. That is, the optical sensor 60A is located around the light source 154 in plan view. Light of the light source 154 reaches the optical sensor 60A through the umbilical fossa 101 of the human body Bd.

Since this configuration increases the light-receiving area of the optical sensor 60A of the detection device 1B according to the first modification of the second embodiment to larger than that of the detection device 1 according to the first embodiment, the sensor sensitivity of the detection device 1B is higher than the detection device 1. The increase in sensor sensitivity can accordingly reduce the power consumption of the light source in the detection device 1B according to the first modification of the second embodiment.

### Second Modification of Second Embodiment

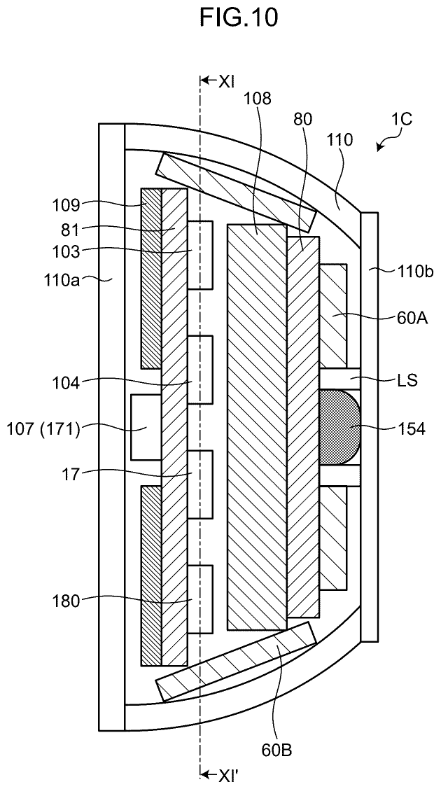

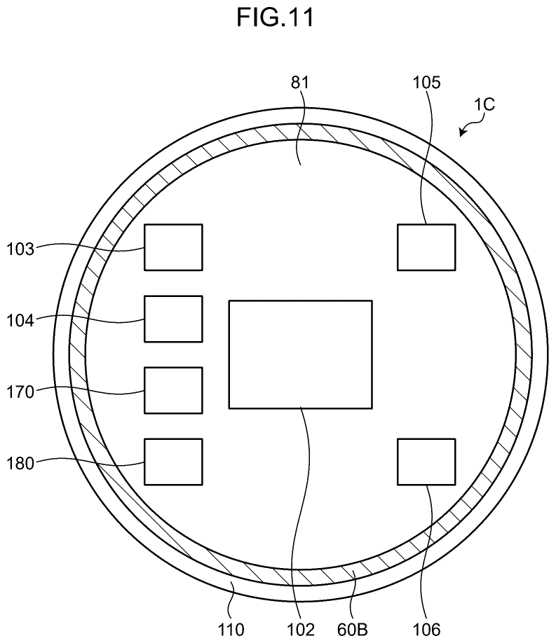

FIG. 10 is a schematic sectional view of a detection device according to a second modification of the second embodiment. FIG. 11 is a plan view of a section along XI-XI′ in FIG. 10. In the following description, the same components as those described in either of the embodiments described above are denoted by the same reference numerals, and the description thereof will not be repeated.

As illustrated in FIG. 10, a detection device 1C according to the second modification of the second embodiment includes the optical sensor 60A as a first optical sensor, in the same way as the detection device 1B according to the first modification of the second embodiment. The detection device 1C according to the second modification of the second embodiment includes a second optical sensor 60B located so as to surround the outer peripheral surface of the battery. The second optical sensor 60B has a shape of a conical frustum.

As illustrated in FIG. 11, the second optical sensor 60B is located so as to surround the control circuit 102, the LED driver 105, the memory 106, the battery driver 180, the charge control driver 170, the acceleration sensor 104, and the temperature sensor 103 in plan view.

Since this configuration further increases the light-receiving area of the detection device 1C according to the second modification of the second embodiment so as to be larger than that of the detection device 1B according to the first modification of the second embodiment, the sensor sensitivity of the detection device 1C is higher than the detection device 1B. The increase in sensor sensitivity can accordingly reduce the power consumption of the light source lower in the detection device 1C according to the second modification of the second embodiment as compared to that of the light source of the detection device 1B according to the first modification of the second embodiment.

The second optical sensor 60B is formed into a fan-shaped ring shape in plan view, and then bent to be formed into a conical frustum shape, thus being flexibly bendable and having a good material quality.

### Third Embodiment

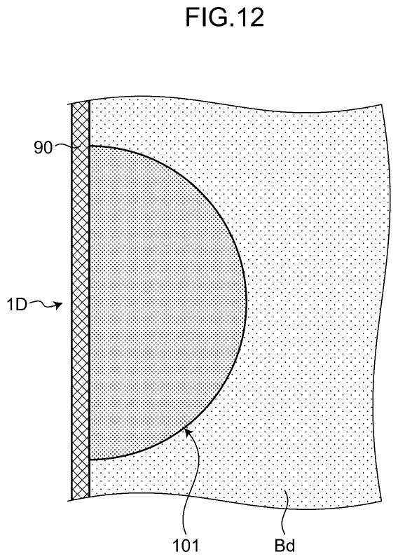

FIG. 12 is a schematic sectional view illustrating a detection device according to a third embodiment in the state of being accommodated in the navel of the human body. In the following description, the same components as those described in either of the embodiments described above are denoted by the same reference numerals, and the description thereof will not be repeated.

In a detection device 1D according to the third embodiment, a light-blocking adhesive layer 90 is provided on the bottom surface 110a of the housing 110. The adhesive layer 90 is a flexible film and is fixed so as to cover the perimeter of the umbilical fossa 101 of the human body Bd.

With this configuration, no protrusions from the human body Bd are present, so that the user can move freely without being restricted in terms of cloths to be worn.

### Fourth Embodiment

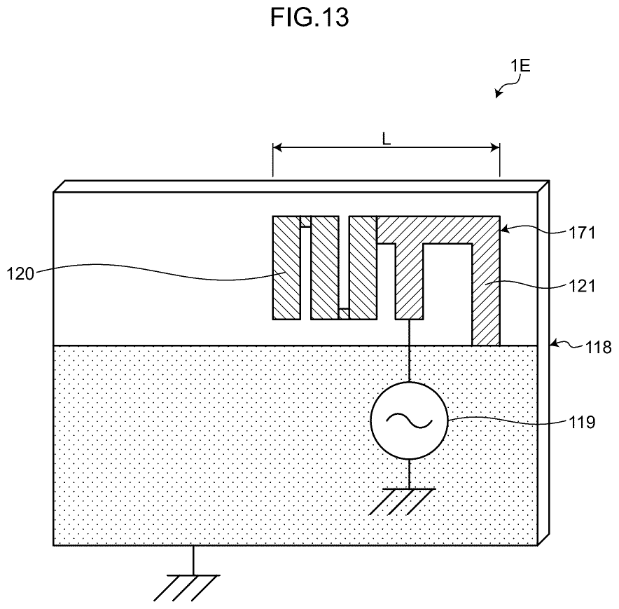

FIG. 13 is a schematic diagram illustrating a configuration example of a communication antenna of a detection device according to a fourth embodiment. In the following description, the same components as those described in any one of the embodiments described above are denoted by the same reference numerals, and the description thereof will not be repeated.

The shape of the communication antenna 171 of a detection device 1E according to the fourth embodiment has at least one of an inverted F-shaped portion 121 or a bent portion 120.

The communication antenna 171 is located on a circuit board 118, and the inverted F-shaped portion 121 is coupled to a ground 119.

An antenna length L that is the length of the communication antenna 171 is calculated by Expression (1) below.

$$L = λ / 4 √ ( ( 1 + {ε}_{r} ) / 2 ) \qquad ( 1 )$$

where λ is a reflection wavelength and εᵣ is relative permittivity.

When the communication frequency of the communication antenna 171 is 2.4 GHz and the relative permittivity Er is 4.6 (equivalent to that of glass epoxy), the antenna length L is approximately 20 mm. When the communication antenna 171 is a near-field communication antenna, the antenna length L may be shorter than 20 mm.

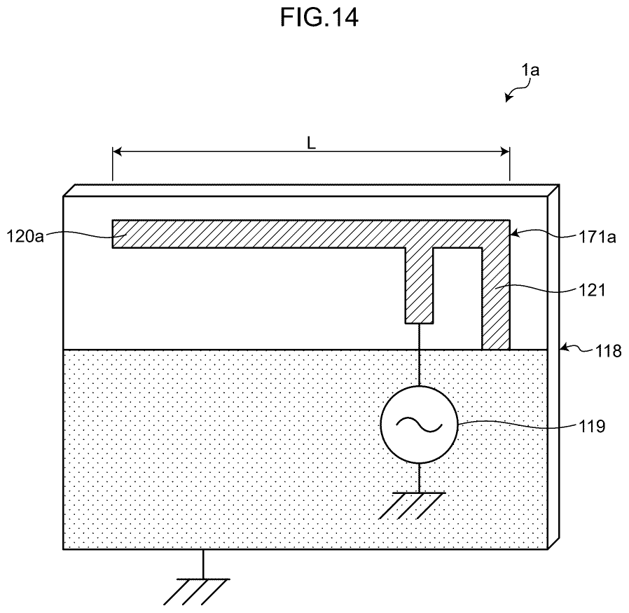

FIG. 14 is a schematic diagram illustrating a configuration example of a communication antenna of a detection device according to a comparative example. In a communication antenna 171a of a detection device 1a according to the comparative example, an end 120a of the communication antenna 171a is not bent, so that the antenna length L is longer than that of the communication antenna 171 of the detection device 1E according to the fourth embodiment. This configuration results in a larger area occupied by the communication antenna 171a.

In contrast, the bent portion 120 of the detection device 1E of the fourth embodiment is two-dimensionally bent with respect to the length direction of the communication antenna 171.

This configuration allows the area occupied by the communication antenna 171 in the detection device 1E according to the fourth embodiment to be reduced to a greater extent than in the detection device 1a according to the comparative example.

### Modification of Fourth Embodiment

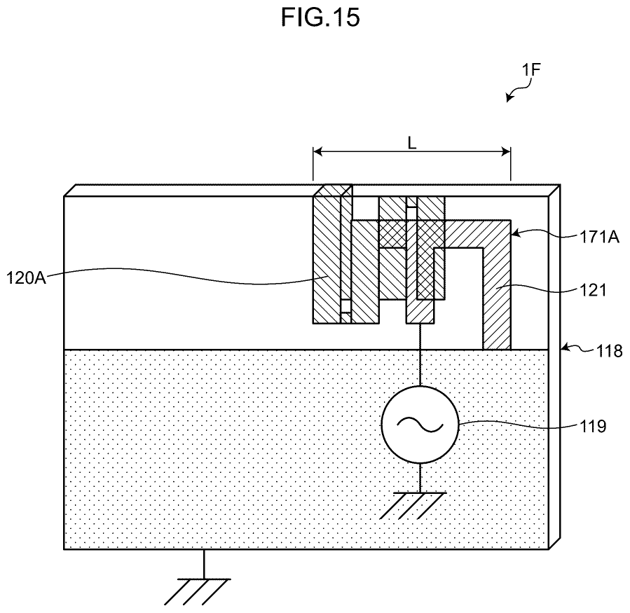

FIG. 15 is a schematic diagram illustrating a configuration example of a communication antenna of a detection device according to a modification of the fourth embodiment. In the following description, the same components as those described in any one of the embodiments described above are denoted by the same reference numerals, and the description thereof will not be repeated.

A bent portion 120A of a detection device 1F according to the modification of the fourth embodiment is three-dimensionally bent with respect to the length and thickness directions of a communication antenna 171A.

This configuration allows the area occupied by the communication antenna 171A in the detection device 1F according to the modification of the fourth embodiment to be reduced to a greater extent than in the detection device 1E according to the fourth embodiment.

### Fifth Embodiment

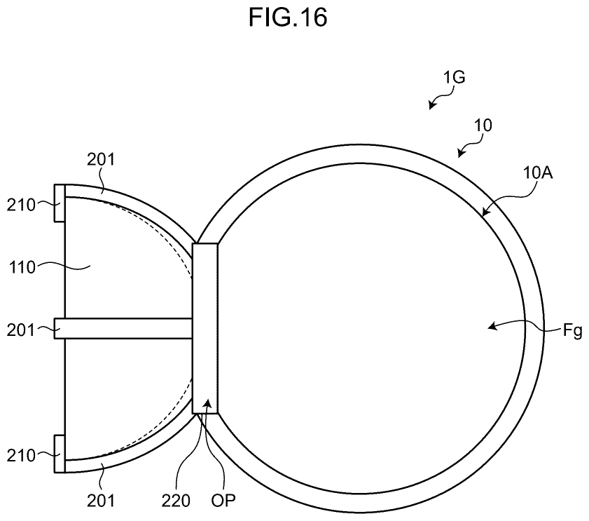

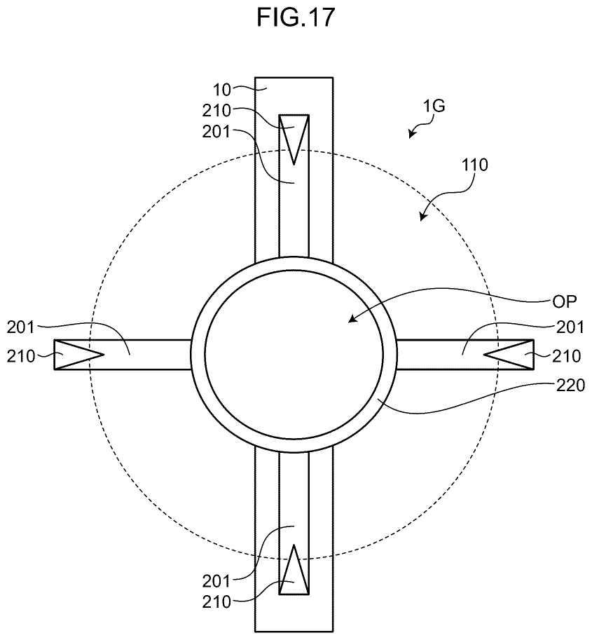

FIG. 16 is a sectional view of a detection device according to a fifth embodiment. FIG. 17 is a plan view of the detection device according to the fifth embodiment. In the following description, the same components as those described in any one of the embodiments described above are denoted by the same reference numerals, and the description thereof will not be repeated.

A detection device 1G according to the fifth embodiment includes a ring 10 wearable on a finger Fg of the human body, the housing 110, supports 201, claws 210, and a pressure-bonding member 220.

As illustrated in FIG. 16, the ring 10 has a hollow portion 10A. The finger Fg of the human body is inserted into the hollow portion 10A of the ring 10, which is worn on the finger Fg while being in contact therewith. Examples of the finger Fg include a thumb, an index finger, a middle finger, a ring finger, and a little finger.

As illustrated in FIGS. 16 and 17, the supports 201 are arranged so as to extend outward from the pressure-bonding member 220 in four directions of upward, downward, leftward, and rightward directions. The dome-shaped housing 110 is accommodated inside the supports 201. The supports 201 have a certain degree of elasticity, and therefore can securely hold the housing 110 therebetween.

The claws 210 are provided at respective ends of the four supports 201. The claws 210 fix the bottom surface 110a of the housing 110.

As illustrated in FIG. 16, the pressure-bonding member 220 that pressure-bonds the apex of the housing 110 is provided between the ring 10 and the housing 110. One end of the ring 10 and each of the supports 201 are fixed to the pressure-bonding member 220.

The pressure-bonding member 220 has an opening OP. The skin of the finger Fg attached to the hollow portion 10A comes in contact with the apex of the housing 110 through the opening OP.

The detection device 1G according to the fifth embodiment can thereby detect the information on the living body in the finger Fg or the like. Examples of the information on the living body include, but are not limited to, the pulse wave, pulsation, and a vascular image of the finger or a palm. That is, the detection device 1G may be configured as a fingerprint detection device that detects fingerprints or a vein detection device that detects vascular patterns of, for example, veins.

While the preferred embodiments of the present disclosure has been described above, the present disclosure is not limited to such embodiments. The content disclosed in the embodiments is merely an example, and can be variously modified within the scope not departing from the gist of the present disclosure. Any modifications appropriately made within the scope not departing from the gist of the present disclosure also naturally belong to the technical scope of the present disclosure. At least one of various omissions, substitutions, and changes of the components can be made without departing from the gist of the embodiments and the modifications thereof described above.

What is claimed is:

1. A detection device comprising:
an optical sensor and a light source; and
a light-transmitting dome-shaped or spherical frustum-shaped housing that covers the optical sensor and the light source, wherein
the optical sensor and the light source are arranged on the same plane in plan view.

2. The detection device according to claim 1, wherein a maximum diameter of the housing is 3 cm or smaller in plan view.

3. The detection device according to claim 2, wherein
the housing comprises a control circuit on a back side of the optical sensor and the light source, and
a charge coil and a communication antenna are arranged on a back side of the control circuit.

4. The detection device according to claim 3, further comprising an acceleration sensor, wherein
the acceleration sensor is located on the same plane as the control circuit.

5. The detection device according to claim 4, wherein a bottom surface of the housing has a light-blocking property.

6. The detection device according to claim 5, wherein the light source comprises a near-infrared light-emitting diode (LED) configured to emit near-infrared light, a red LED configured to emit red light, and a green LED configured to emit green light.

7. The detection device according to claim 6, wherein a light-blocking adhesive layer is provided on the bottom surface of the housing.

8. The detection device according to claim 7, wherein the charge coil is located so as to be wound around the communication antenna in plan view.

9. The detection device according to claim 8, wherein the light source is located around the optical sensor in plan view.

10. The detection device according to claim 8, wherein the optical sensor is located around the light source in plan view.

11. The detection device according to claim 10, comprising a battery between the control circuit and both the optical sensor and the light source, wherein
the optical sensor comprises a first optical sensor located on the same plane as the light source in plan view and a second optical sensor located so as to surround an outer peripheral surface of the battery.

12. The detection device according to claim 9, wherein a shape of the communication antenna has at least one of an inverted F-shaped portion or a bent portion.

13. The detection device according to claim 12, comprising a ring wearable on a finger, wherein
a pressure-bonding member to pressure-bond an apex of the housing is provided between the ring and the housing.

14. The detection device according to claim 13, wherein the optical sensor is an organic photodiode (OPD).

15. The detection device according to claim 14, wherein the control circuit is configured to obtain an amount of activity based on acceleration data of the acceleration sensor.

16. The detection device according to claim 15, wherein the control circuit is configured to measure at least one of a pulse frequency or a blood oxygen level based on detection data of the optical sensor.

17. The detection device according to claim 16, further comprising a temperature sensor, wherein
the control circuit is configured to acquire body temperature data from the temperature sensor.
